(19) United States
(12) Patent Application Publication
(10) Pub. No.: US 2023/0226007 A1
(43) Pub. Date: Jul. 20, 2023

(54) NANOPARTICULATE COMPOSITION

(71) Applicant: Algiax Pharmaceuticals GmbH, Erkrath (DE)

(72) Inventors: Birgit Hasse, Velbert (DE); Guido Koopmans, JC Sittard (NL); Lena Liebich, Lörrach (DE); Ansgar Bögershausen, Freiburg (DE); Sandra Kneisel, Gottenheim (DE); Martin Hagedorn, Breisach (DE); Matthias Rischer, Frankfurt (DE)

(21) Appl. No.: 18/008,421

(22) PCT Filed: Jun. 11, 2021
(86) PCT No.: PCT/EP2021/065757

§ 371 Date: Dec. 5, 2022

(30) Foreign Application Priority Data
Jun. 26, 2020 (EP) 20182425.7

(51) Int. Cl. A61K 31/277; A61K 9/51; A61K 9/48

(52) U.S. Cl. CPC A61K 31/277; A61K 9/5161; A61K 9/5146; A61K 9/485; A61K 9/4866; A61K 9/4858

(57) ABSTRACT

A nanoparticulate composition including particles comprising at least one active ingredient, wherein the particles have an effective average particle size is in the range of about 70 nm to about 220 nm measured by laser light scattering method, wherein at least 50% of said active agent particles have a particle size, by weight (volume based), of less than the effective average particle size; and (b) at least one surface stabilizer and/or at least one polymeric stabilizer, wherein the composition includes (aa) particles of at least one active ingredient selected from the group consisting of (Z)-2-cyano-3-cyclopropyl-3-hydroxy-N-(3-methyl-4-(trifluoromethyl)phenyl) prop-2-enamide, (Z)-2-cyano-3-hydroxy-N-[4-(trifluoromethyl)phenyl]hept-2-en-6-ynamide, 2-cyano-3-cyclopropyl-N-(4-fluorophenyl)-3-hydroxyacrylamide, derivatives thereof, salts thereof and pro-drugs thereof, wherein the particles have an effective average particle size of less than about 2000 nm; and (bb) at least one surface stabilizer and/or at least one polymeric stabilizer.

### CROSS REFERENCE TO RELATED APPLICATIONS

This is a US national phase under 35 USC § 371 of international patent application no. PCT/EP2021/065757, filed 11 Jun. 2021, which claims priority to European application no. EP 20182424.7, filed Jun. 26, 2020; the entire content of each is herein incorporated by reference in its entirety.

### FIELD OF THE INVENTION

The present disclosure relates to a pharmaceutical composition comprising an active ingredient having an improved bioavailability and onset of action. Furthermore, the present invention relates to a pharmaceutical composition comprising said composition and the use of said composition for the preparation of a medicament Finally, the present invention relates to a method of making said composition and a system for the treatment of certain diseases using said composition.

### BACKGROUND OF THE INVENTION

### A. Background Regarding Nanoparticulate Compositions

U.S. Pat. No. 5,145,684 describes nanoparticulate compositions comprising particles of a poorly soluble therapeutic or diagnostic agent. A non-crosslinked surface stabilizer is associated with the surface thereof.

Methods of making nanoparticulate compositions are described, for example, in U.S. Pat. Nos. 5,518,187 and 5,862,999, both for “Method of Grinding Pharmaceutical Substances;” U.S. Pat. No. 5,718,388, for “Continuous Method of Grinding Pharmaceutical Substances;” and U.S. Pat. No. 5,510,118 for “Process of Preparing Therapeutic Compositions Containing Nanoparticles.”

There are a number of nanoparticulate formulations listed under several patents for specific active pharmaceutical ingredients. WO 03/103633 describes nanoparticulate compositions comprising one or more sterols or stanols such as sitosterol or phytosterol. US 2008/02133374 A1 describes nanoparticulate compositions comprising sorafenib or a salt of sorafenib. US 2009/0238867 A1 describes nanoparticulate compositions comprising anidulafungin. U.S. 2008/0317843 A1 is directed to nanoparticulate compositions comprising modafinil. EP 1 658 053 describes novel nanoparticulate compositions of Sildenafil free base. WO 03/066021 A2 is directed to nanoparticulate compositions comprising lysozyme as a surface stabilizer. Another WO patent application is directed to nanoparticulate compositions comprising at least one poorly soluble MAP Kinase inhibitor and at least one surface stabilizer. US 2008/00220075 A1 describes nanoparticulate compositions comprising at least one poorly soluble angiogenesis inhibitor and at least one surface stabilizer. US 2003/0224058 A1 and US 2008/0241070 A1 are directed to fibrate compositions and their in-vivo behaviour in which the fibrate particles have an average particle size of less than about 2000 nm. US 2005/0095297 A1 discloses nanoparticulate formulations or suspensions comprising fibrate and Vitamin E TPGS. US 2019/0117674 A1 is directed to nanoparticulate compositions of Ganaxolone with a mean diameter between 50 and 500 nm. US 2018/0228810 A1 discloses nanoparticulate compositions comprising mexoxicam particles having a particle size of less than about 2000 nm. US 2018/0008601 A1 is directed to nanoparticulate forms of piperazine compounds. US 2015/0320682 A1 is directed to nanoparticulate compositions comprising megestrol. US 2013/0243830 A1 relates to nanoparticulate compositions containing corticosteroid compounds.

U.S. Pat. No. 6,316,029 B1 is directed to rapidly disintegrating solid oral dosage forms of a poorly soluble active ingredient and at least one pharmaceutical acceptable water-soluble or water-dispersible excipient wherein the poorly soluble active ingredient particles have an average diameter of less than 2000 nm.

WO 03/024424 A1 and US 2012/0087984 A1 describe methods for stabilizing active ingredients, particularly pharmaceutical ingredients by forming active ingredients into a nanoparticulate composition comprising the active ingredient and at least one surface stabilizer.

WO 02/094215 A2 is directed to nanoparticulate compositions of poorly soluble drugs and at least one copolymer of vinyl pyrrolidone and vinyl acetate as a surface stabilizer adsorbed on the surface of the drug.

US 2002/0110597 A1 and U.S. Pat. No. 6,375,986 B1 disclose solid nanoparticulate compositions comprising of a poorly soluble active agent, at least one polymeric surface stabilizer and dioctyl sodium sulfosuccinate (DOSS).

US 20020012675 is directed to controlled release of nanoparticulate compositions comprising a nanoparticulate agent and a rate-controlling polymer, which releases the agent after administration between 2 and 24 hours or longer.

### B. Background Regarding (Z)-2-cyano-3-cyclopropyl-3-hydroxy-N-(3-methyl trifluoromethyl)phenyl) prop-2-enamide (AP-325—INN: Laflunimus)

(Z)-2-cyano-3-cyclopropyl-3-hydroxy-N-(3-methyl-4-trifluoromethyl)phenyl) prop-2-enamide (in the following also Laflunimus (INN) or AP-325 (working name)) and its derivatives belong to a class of compounds which are useful in the treatment of central nervous system (CNS)-trauma related disorders. Other compounds with such an activity are described in US 2016/022688 A1.

AP-325 (Laflunimus) has the following structure according to formula I:

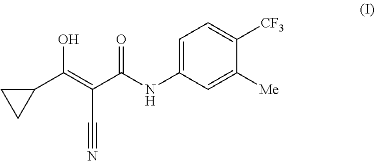 [SMILES: Cc1cc(NC(=O)/C(C#N)=C(\O)C2CC2)ccc1C(F)(F)F]

Similar useful compounds, which may also be understood as derivatives of AP-325, are:

(Z)-2-cyano-3-hydroxy-N-[4-(trifluoromethyl)phenyl]hept-2-en-6-ynamide (FK778—INN: Manitimus) according to the following formula II

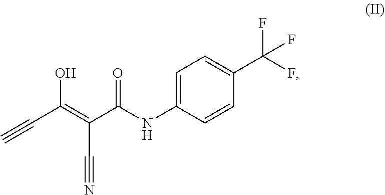 [SMILES: C#C/C(O)=C(\C#N)C(=O)Nc1ccc(C(F)(F)F)cc1]

and 2-cyano-3-cyclopropyl-N-(4-fluorophenyl)-3-hydroxyacrylamide according to the following formula III:

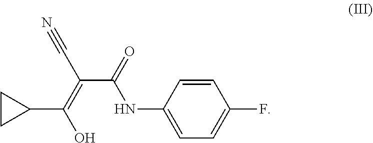 [SMILES: N#C/C(C(=O)Nc1ccc(F)cc1)=C(/O)C1CC1]

AP-325 is a pharmaceutical agent, which is currently in clinical development. Based on the solubility of the drug (see FIG. 3), AP-325 can be considered as practically insoluble at a physiological pH of 6.8 (phosphate buffer). For a better characterization of active ingredients with low solubility, a classification according to the BCS system (=Biopharmaceutics Classification System) was introduced (Guidance for Industry: Immediate release solid oral dosage forms and FDA, 1995). This BCS system distinguishes four categories based on the solubility and permeability of the active substance. At a pH value of below 7 AP-325 can be classified as a BCS class II drug substance. The low solubility of AP-325 negatively affects the absorption into the body after oral administration due to a limited absorption of the active agent in the small intestine (such as the jejunum or ileum), which is an essential absorption window for the uptake of a drug. Consequently, the therapeutic effect cannot be achieved.

### FURTHER PRIOR DISCLOSURES

US2010/297252 A1 is directed to nanoparticulate compositions comprising meloxicam particles having an effective average particle size of less than about 2000 nm.

EP 2 632 451 A2 relates to compounds and pharmaceutical compositions for use in the treatment of neuropathic pain and the neuropathic pain syndromes.

### BRIEF SUMMARY OF THE INVENTION

The present invention relates in particular to a nanoparticulate composition comprising particles of at least one active ingredient selected from the group consisting of (Z)-2-cyano-3-cyclopropyl-3-hydroxy-N-(3-methyl-4-trifluoromethyl)phenyl) prop-2-enamide (Laflunimus, AP-325) in form of the free base, (Z)-2-cyano-3-hydroxy-N-[4-(trifluoromethyl)phenyl]hept-2-en-6-ynamide, 2-cyano-3-cyclopropyl-N-(4-fluorophenyl)-3-hydroxyacrylamide, derivatives thereof, salts thereof and pro-drugs thereof, wherein the particles have an effective average particle size of less than about 2000 nm. The composition of the invention comprises preferably at least one surface stabilizer and/or at least one polymeric stabilizer.

Thus, the present invention essentially relates to the following three chemical compounds or active ingredients:
(Z)-2-cyano-3-cyclopropyl-3-hydroxy-N-(3-methyl-4-(trifluoromethyl)phenyl) prop-2-enamide according to above formula I, also known as Laflunimus (INN) and preferably designated as AP-325 in the present patent specification; and/or
(Z)-2-cyano-3-hydroxy-N-[4-(trifluoromethyl)phenyl]hept-2-en-6-ynamide, also known as Manitimus (INN) and preferably designated as FK778 in the present patent specification; and/or
2-cyano-3-cyclopropyl-N-(4-fluorophenyl)-3-hydroxyacrylamide

Furthermore, the present invention also relates to derivatives of the above compounds or active ingredients, salts and pro-drugs thereof.

The compositions of the invention show a surprisingly substantially improved bioavailability and on-set of action of the at least one active ingredient compared to a pharmaceutical dosage form comprising the active pharmaceutically ingredient having a particle size above 2 microns.

Another aspect of the invention is directed to pharmaceutical compositions comprising the above described composition in combination with at least one pharmaceutically acceptable excipient.

Preferably, the compositions also comprise at least one surface stabilizer and/or at least one polymeric stabilizer associated with the particles of the active ingredient.

This invention is also directed to a method of making the above described composition. Such a method may comprise contacting particles of the at least one active ingredient with at least one surface stabilizer and/or at least one polymeric stabilizer for a time and under conditions sufficient to provide a composition comprising particles of the active ingredient having an effective average particle size of less than about 2 microns. The one or more surface stabilizers and/or the one or more polymeric stabilizers can be contacted with the active ingredient either before, preferably during, or after particle size reduction.

This invention is also directed to a method of making a solid oral dosage form in which the nanoparticles from the nanosuspensions containing the at least one active ingredient as described for the above composition are bound on a suitable pharmaceutical excipient or carrier by using a fluid bed drying process, a spray drying process, an extrusion process or a granulation process.

This invention also relates to a pharmaceutical system for the treatment of certain diseases in a subject comprising administering to a subject of an effective amount of the above described composition.

This invention also relates to a pharmaceutical system containing particles of at least one active ingredient, e.g. AP-325, having an effective average particle size of less than about 2 microns which show a significant increase of the AUC (Area Under the Curve) of the active ingredient, e.g. AP-325, in the blood plasma and thereof a significant increase of the bioavailability compared to pharmaceutical systems containing particles of the active ingredient, e.g. AP-325, having an average particle size above 2 microns.

This invention is further directed to a pharmaceutical system containing particles of at least one active ingredient, e.g. AP-325, having an effective average particle size of less than about 2 microns which show a significant decrease of the Tₘₐₓ of the active ingredient, e.g. AP-325, in the blood plasma and thereof a significant faster on-set of action of the active ingredient, e.g. AP-325, compared to pharmaceutical systems containing particles of the active ingredient, e.g. AP-325, having an average particle size above 2 microns.

### BRIEF DESCRIPTION OF THE DRAWINGS

FIG. 1a: In-vitro-release dissolution profiles of a nanoparticulate composition (capsule, example 7) of AP-325 over a period of 24 months at 25° C./60% r.h. The dissolution data confirm a very good stability and fulfil the relevant requirements of the Ph. Eur. for oral dosage forms.

FIG. 1b: Stability data of capsule batch G0625K102 stored in HDPE bottles over a period of 24 months at 25° C./60% r.h. The stability data demonstrate that the AP-325 nanoparticulate composition is unaffected under the ICH storage conditions to any changes of the investigated parameters and can be regarded as very stable over the investigated storage period.

FIG. 2: Structural formulas of AP-325 and its derivatives.

FIG. 3: Solubility of AP-325 in different solvents at ambient temperature. AP-325 can be considered as practically insoluble at a physiological pH of 6.8 (phosphate buffer).

FIG. 4a: Nanoparticulate compositions of AP-325 applied in pk study in rats (study 1).

FIG. 4b: Nanoparticulate compositions of AP-325 applied in pk study in rats (study 2).

FIG. 5a: Tabulated results of pk study in rats (study 1). The nanoparticulate compositions of AP-325 exhibit increased bioavailability, at the same dose. Significant higher AUC and Cmax values for the preferable nanoparticulate composition (N014) of AP-325 can be reached compared to the microparticulate composition of AP-325 for the same dose.

FIG. 5b: Tabulated results of pk study in rats (study 2). Most nanoparticulate compositions have a significant faster Tmax (less than 2 h) than the composition containing AP-325 particles above 2 microns (Mikro N064). The nanoparticulate compositions of AP-325 out-performs their microparticulate counterparts on the pharmacokinetic parameters Tmax, Cmax and AUC.

FIG. 6: Tabulated results of pk study in dogs. The nanoparticulate composition showed a Tmax of 2 h whereas the composition containing AP-325 particles above 2 microns showed a Tmax of 2.5 h.

FIG. 7: Nanoparticulate compositions of AP-325 capsules applied in a pk study demonstrating a fast on-set of action and a dose-increasing good absorption in humans.

FIG. 8: XRPD data of dried AP-325 Nanosuspension, Bottom-line: Placebo, Lower middle-line: AP-325, Upper-line: Nanoparticulate formulation of AP-325, Upper middle-line: AP-325 suspended. All X-ray patterns remained unchanged after the milling process, proving that the crystallinity of the active ingredient AP-325 has not changed.

FIG. 9: Comparison of the particle size distribution (PSD) of G0625C101 at 25° C./60% r.h. (granules/intermediate product of G0625K102). The PSD remained stable at storage conditions of 25° C./60% r.h. over a period of 24 months representing a very good stability of the nanoparticles of AP-325 in the dosage form.

FIG. 10: Comparison of the particle size distribution of G0625C101 at 40° C./75% r.h. (granules/intermediate product of G0625K102). The PSD remained stable at storage conditions of 40° C./75% r.h. over a period of 6 months representing a very good stability of the nanoparticles of AP-325 in the dosage form.

### DETAILED DESCRIPTION OF THE INVENTION

### A. Introduction to the Disclosure

The following provides an introduction to the disclosure with certain preferred embodiments to follow. The disclosure includes a nanoparticulate composition, which includes (a) particles having at least one active ingredient, where the particles have an effective average particle size of less than about 2000 nm; and (b) at least one surface stabilizer and/or at least one polymeric stabilizer.

In some embodiments, the composition includes (a) particles of at least one active ingredient selected from the group consisting of (Z)-2-cyano-3-cyclopropyl-3-hydroxy-N-(3-methyl-4-(trifluoromethyl)phenyl) prop-2-enamide, (Z)-2-cyano-3-hydroxy-N-[4-(trifluoromethyl)phenyl]hept-2-en-6-ynamide, 2-cyano-3-cyclopropyl-N-(4-fluorophenyl)-3-hydroxyacrylamide, derivatives thereof, salts thereof and pro-drugs thereof, wherein the particles have an effective average particle size of less than about 2000 nm; and (b) at least one surface stabilizer and/or at least one polymeric stabilizer.

In some embodiments, the effective average particle size is less than 1400 nm, preferably less than 900 nm, more preferably less than 300 nm, in particular wherein the effective average particle size is more than 40 nm, preferably more than 50 nm, more preferably more than 60 nm.

In some embodiments the effective average particle size is in the range from about 70 nm to about 220 nm, preferably in the range from about 90 nm to about 210 nm, more preferably in the range from about 100 nm to about 200 nm.

In some embodiments the composition is a pharmaceutical formulation which is selected from the group consisting of oral tablets, capsules, sachets, stick packs, buccal, topical dosage forms or liquid dispersions and gels.

In some embodiments the composition includes one or more pharmaceutically acceptable excipients, carriers, or a combination thereof.

In some embodiments, the at least one active ingredient is present in an amount in the range from about 99.5% to about 0.001%, preferably in the range from about 95% to about 0.1%, and more preferably in the range from about 90% to about 0.5%, by weight, based on the total combined dry weight of the active ingredient and the at least one surface stabilizer and/or polymeric stabilizer, not including other excipients.

In some embodiments, at least one surface stabilizer and/or the at least one polymeric stabilizer is present in an amount in the range from about 0.5% to about 99.9% by weight, preferably in the range from about 5.0% to about 99.9% by weight, and more preferably from about 10% to about 99.5% by weight, based on the total combined dry weight of the active ingredient and the at least one surface and/or the at least one polymeric stabilizer, not including other excipients. In some embodiments, the at least one surface stabilizer is selected from the group consisting of an anionic surface stabilizer, a cationic surface stabilizer, a zwitterionic surface stabilizer, a nonionic surface stabilizer, and an ionic surface stabilizer. In some embodiments, the at least one surface stabilizer comprises or consists of sodium glycocholate. In some embodiments the at least one polymeric stabilizer is selected from the group consisting of cellulose derivatives, polysaccharides, polyethylene derivatives, phospholipids, alginates and the like. In some embodiments, the at least one surface stabilizer and/or the at least one polymeric stabilizer is/are selected from the group consisting of hydroxypropyl methylcellulose, hydroxypropylcellulose, sodium lauryl sulfate, gelatin, casein, lecithin and other phosphatides, bile salts like taurocholate and derived salts like sodium glycocholate, dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, the commercially available Tweens such as e.g., Tween 20® and Tween 80®, polyethylene glycols, polyoxyethylene stearates, colloidal silicon dioxide, phosphates, carboxymethylcellulose calcium, carboxymethylcellulose sodium, methylcellulose, hydroxyethylcellulose, hydroxypropylmethylcellulose phthalate, noncrystalline cellulose, magnesium aluminium silicate, triethanolamine, polyvinyl alcohol, poloxamers, e.g., Pluronics F68® and F127®, which are block copolymers of ethylene oxide and propylene oxide; poloxamines, e.g. Tetronic 908®, also known as Poloxamine 908®, which is a tetrafunctional block copolymer derived from sequential addition of propylene oxide and ethylene oxide to ethylenediamine, Tritons X-200® which is an alkyl aryl polyether sulfonate; Crodestas F-110®, which is a mixture of sucrose stearate and sucrose distearate, n-alkyl-beta-D-glucopyranosides; PEG-phospholipid, PEG-cholesterol, PEG-cholesterol derivative, PEG-vitamin A, PEG-vitamin E e.g. TPGS-1000®, lysozyme, and the like. In some embodiments, the at least one polymeric stabilizer is selected from poloxamer and/or hydroxypropylcellulose.

In some embodiments, the composition includes one or more carrier agents, binding agents, filling agents, lubricating agents, suspending agents, sweeteners, flavouring agents, buffers, wetting agents, disintegrants, effervescent agents, and other excipients.

In some embodiments, the composition includes one or more carrier agents selected from the group consisting of mannitol, lactose, lactose monohydrate, cellulose, cellulose derivatives or isomalt.

In some embodiments, the composition includes one or more filling agents and/or one or more diluent agents, where the filling agents and the diluent agents are selected from the group consisting of mannitol, lactose monohydrate, lactose anhydrous, starches, dibasic calcium phosphate, saccharides and mixtures thereof; and/or one or more binding agents which are selected from the group consisting of celluloses and cross-linked polyvinylpyrrolidone, microcrystalline cellulose, such as Avicel® PH101 and Avicel® PH102, and silicified microcrystalline cellulose (ProSolv SMCCT™).

In some embodiments, the composition includes one or more lubricants which are selected from the group consisting of colloidal silicon dioxide, such as Aerosil® 200, talc, stearic acid, magnesium stearate, calcium stearate, and silica gel.

In some embodiments the composition includes comprises talc.

In some embodiments the composition includes one or more disintegrants which are selected from the group consisting of crosslinked polyvinylpyrrolidone, corn starch, potato starch, maize starch and modified starches, croscarmellose sodium, cross-povidone, sodium starch glycolate, and mixtures thereof.

In some embodiments the composition includes comprises one or more effervescent agents which are selected from the group consisting of citric acid, tartaric acid, malic acid, fumaric acid, adipic acid, succinic acid, alginic acid and acid salts thereof, sodium carbonate, sodium bicarbonate, potassium carbonate, potassium bicarbonate, magnesium carbonate, sodium glycine carbonate, L-lysine carbonate, and arginine carbonate.

In some embodiments the composition includes one or more sweeteners which are selected from the group consisting of sucrose, xylitol, sodium saccharin, cyclamate, aspartame, and acesulfame.

In some embodiments the composition is provide with at least one pharmaceutically acceptable excipient.

The disclosure also provides the use of a pharmaceutical composition for the preparation of a medicament.

The disclosure also provides a method of making the composition, which includes contacting particles of the at least one active ingredient with at least one surface stabilizer and/or at least one polymeric stabilizer for a time and under conditions sufficient to provide a composition including particles of the active ingredient having an effective average particle size as defined herein.

In some embodiments, the step of contacting includes grinding, wet grinding, high pressure homogenization, homogenization, emulsion techniques, supercritical fluid particle generation techniques, precipitation or a combination thereof.

A method of making a solid oral dosage form in which the nanoparticles from the nanosuspensions containing one or several of the active ingredients as itemed above is bound on a suitable pharmaceutical excipient or carrier is also provided by using a fluid bed drying process, a spray drying process, an extrusion process or a granulation process.

In some embodiments, the formulation of nanoparticles having at least one of the mentioned active ingredients are formulated into tablets, capsules, sachets or stick packs.

Also disclosed is a system for use in the treatment or the prevention of neuropathic pain and/or central nervous system trauma related disorder and/or certain other diseases in a subject, which includes administering to a subject of an effective amount of the composition.

In some embodiments, the nanoparticulate composition has a reduced Tₘₐₓ, a higher cₘₐₓ and higher AUC in mammalian subjects compared to a composition containing the one or several active ingredients from item 2 having an average effective particle size of more than about 2000 nm.

In some embodiments, the nanoparticulate composition does not have a disintegration time of less or equal to 3 min.

In some embodiments, the pharmaceutical composition includes one or more additional active agents useful for the treatment of certain human diseases.

In some embodiments, the nanoparticulate composition does not have a disintegration time of less or equal to 3 min.

In some embodiments, the system is used for the treatment of wherein the compound is used in the treatment of peripheral and/or predominantly peripheral neuropathic pain or central and/or predominantly central neuropathic pain. In some embodiments, the predominantly peripheral neuropathic pain is of a type that is selected from the following types of neuropathic pain and/or has a cause that is selected from the group of the following causes: systemic diseases, e.g. diabetic neuropathy; drug-induced lesions, e.g. neuropathy due to chemotherapy; traumatic syndrome and entrapment syndrome; lesions in nerve roots and posterior ganglia; neuropathies after HIV infections; neuralgia after Herpes infections; nerve root avulsions; cranial nerve lesions; cranial neuralgias, e.g., trigeminal neuralgia; neuropathic cancer pain; phantom pain; compression of peripheral nerves, neuroplexus and nerve roots; paraneoplastic peripheral neuropathy and ganglionopathy; complications of cancer therapies, e.g. chemotherapy, irradiation, and surgical interventions; complex regional pain syndrome; type I lesions (previously known as sympathetic reflex dystrophy); and type II lesions (corresponding approximately to causalgia); or whereby the predominantly central neuropathic pain is of a type that has a cause that is selected from the following group of causes: cerebral lesions that are predominantly thalamic; infarction, e.g. thalamic infarction or brain stem infarction; cerebral tumors or abscesses compressing the thalamus or brain stem; multiple sclerosis; head pain syndrome caused by central pain mechanisms like in migraine or migraine pain; brain operations, e.g. thalamotomy in cases of motoric disorders; spinal cord lesions; spinal cord injuries; spinal cord operations, e.g. anterolateral cordotomy; ischemic lesions; anterior spinal artery syndrome; Wallenberg's syndrome; and syringomyelia.

In some embodiments, the neuropathic pain is postherpetic neuralgia (caused by Herpes Zoster), root avulsions, painful traumatic mononeuropathy, painful polyneuropathy (particularly due to diabetes), central pain syndromes (potentially caused by virtually any lesion at any level of the nervous system), postsurgical pain syndromes (eg, postmastectomy syndrome, postthoracotomy syndrome, phantom pain), complex regional pain syndrome (reflex sympathetic dystrophy and causalgia), and/or migraine or migraine pain. In some embodiments, the neuropathic pain is a central pain syndrome caused by spinal cord injury and/or spinal cord contusion. In some embodiments, the neuropathic pain is a chronic neuropathic pain.

In some embodiments, the system is used to treat an inflammatory disease. In some embodiments, the inflammatory disease is selected from the group consisting of metabolic syndrome, and autoimmune disease such as type I diabetes (TID), rheumatoid arthritis, systemic lupus erythematosus, myasthenia gravis, and multiple sclerosis, in particular to treat diabetes.

In some embodiments, system is used to treat type I diabetes and/or type II diabetes.

Also disclosed is a method for treating neuropathic pain and/or neuropathic pain syndromes and/or certain other diseases in a subject in a patient, which includes administering to a subject of an effective amount of the composition or pharmaceutical composition.

In some embodiments, the patient is a human.

In some embodiments, the neuropathic pain syndrome is postherpetic neuralgia (caused by Herpes Zoster), root avulsions, painful traumatic mononeuropathy, painful polyneuropathy (particularly due to diabetes), central pain syndromes (potentially caused by virtually any lesion at any level of the nervous system), postsurgical pain syndromes (eg, postmastectomy syndrome, postthoracotomy syndrome, phantom pain), complex regional pain syndrome (reflex sympathetic dystrophy and causalgia), and/or migraine or migraine pain.

In some embodiments, the neuropathic pain is a central pain syndrome caused by spinal cord injury and/or spinal cord contusion.

In some embodiments, the type of neuropathic pain is selected from those that have a cause that is selected from the group of the following causes: systemic diseases, e.g. diabetic neuropathy; drug-induced lesions, e.g. neuropathy due to chemotherapy; traumatic syndrome and entrapment syndrome; lesions in nerve roots and posterior ganglia; neuropathies after HIV infections; neuralgia after Herpes infections; nerve root avulsions; cranial nerve lesions; cranial neuralgias, e.g., tri-geminal neuralgia; neuropathic cancer pain; phantom pain; compression of peripheral nerves, neuroplexus and nerve roots; paraneoplastic peripheral neuropathy and ganglionopathy; complications of cancer therapies, e.g. chemotherapy, irradiation, and surgical interventions; complex regional pain syndrome; type I lesions (previously known as sympathetic reflex dystrophy); and type II lesions (corresponding approximately to causalgia); migraine and migraine pain; cerebral lesions that are predominantly thalamic; infarction, e.g. thalamic infarction or brain stem infarction; cerebral tumors or abscesses compressing the thalamus or brain stem; multiple sclerosis; brain operations, e.g. thalamotomy in cases of motoric disorders; spinal cord lesions; spinal cord injuries; spinal cord operations, e.g. anterolateral cordotomy; ischemic lesions; anterior spinal artery syndrome; Wallenberg's syndrome; and syringomyelia. In some embodiments, the neuropathic pain is a chronic neuropathic pain.

In some embodiments, the method is used to treat an inflammatory disease. In some embodiments, the inflammatory disease is selected from the group consisting of metabolic syndrome, and autoimmune disease such as type I diabetes (TID), rheumatoid arthritis, systemic lupus erythematosus, myasthenia gravis, and multiple sclerosis, in particular to treat diabetes.

In some embodiments, the method is used to treat type I diabetes and/or type II diabetes.

### B. Additional Preferred Characteristics of the AP-325 Nanoparticulate Compositions of the Invention

### 1. Fast On-Set of Activity

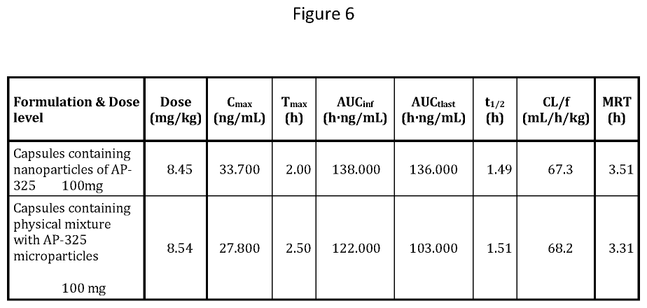

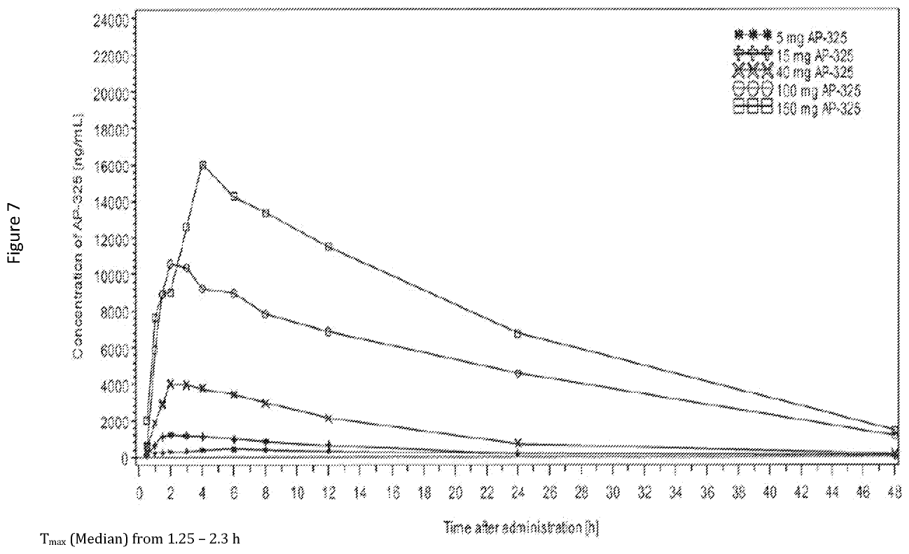

Nanoparticulate compositions of AP-325 show a fast on-set of action, especially compared to compositions of AP-325 which contain particles in the microns range above 2 microns. As demonstrated in figure Sb three of the tested nanoparticulate compositions in rats have a significant faster Tₘₐₓ (less than 2 h) than the composition containing AP-325 particles above 2 microns and only one nanoparticulate composition had the same Tₘₐₓ in this study (about 2 h). This observation was confirmed in the dog study presented in FIGS. 6 and 7 in which the nanoparticulate composition showed a Tₘₐₓ of 2 h whereas the composition containing AP-325 particles above 2 microns showed a Tₘₐₓ of 2.5 h. Finally, the fast on-set of action has been also demonstrated for the nanoparticulate composition of AP-325 in a pk study in humans (see FIG. 7) in which a Tₘₐₓ (median) between 1.25-2.3 h was seen.

The demonstrated fast on-set of action is important in the treatment of the mentioned neuropathic pain and central nervous system trauma related disorder to represent a high level of compliance towards the patients.

### 2. Increased Bioavailability

The nanoparticulate compositions of AP-325 of the invention preferably exhibit increased bioavailability, at the same dose, and require smaller doses as compared to prior non-nanoparticulate AP-325 compositions. This was demonstrated in figure Sa) and Sb) for the nanoparticulate composition of AP-325 (N014/N063) to be superior over the non-nanoparticulate composition of AP-325 (N064/N059) with significant higher bioavailability of 147% in the nanoparticulate composition compared to 82% in the non-nanoparticulate composition of AP-325 and significant higher AUC and Cₘₐₓ values for the preferable nanoparticulate composition (N014) of AP-325 compared to the non-nanoparticulate composition of AP-325 for the same dose.

### 3. Pharmacokinetic Profiles of the AP-325 Nanoparticulate Compositions of the Invention

The present invention provides nanoparticulate AP-325 compositions having a desirable pharmacokinetic profile when administered to mammalian subjects. The desirable pharmacokinetic profile can include one or more of the following characteristics: (1) the Tₘₐₓ of an administered dose of a nanoparticulate AP-325 composition can be less than that of a non-nanoparticulate AP-325 composition, (2) the Cₘₐₓ of a nanoparticulate AP-325 composition can be greater than the Cₘₐₓ of a non-nanoparticulate AP-325 composition, (3) the AUC of a nanoparticulate AP-325 composition can be greater than the AUC of a non-nanoparticulate AP-325 composition. This has been proven in comparative studies reported in figures Sa), Sb) and 7.

In a pk study with healthy volunteers with doses of 5 to 150 mg per patient a Tₘₐₓ (median) from 1.25 h (5 mg) to 2.3 h (150 mg), a Cmax (mean) from 475 ng/ml (5 mg) to 17961 ng/ml (150 mg) and an AUC of 10578 h·ng/mL (5 mg) to 381961 h·ng/mL (5 mg) was seen as shown in FIG. 7, demonstrating the fast on-set of action and a dose-increasing good absorption profile in human for this nanoparticulate composition of AP-325.

### C. Compositions

### 1. AP-325 Derivatives

AP-325 and further chemical compounds, which may be understood as AP-325 derivatives, are described in the Background regarding AP-325 above. Derivative (Z)-2-cyano-3-hydroxy-N-[4-(trifluoromethyl)phenyl]but-2-enamide/Teriflunomide with its shown chemical structure is excluded from this patent:

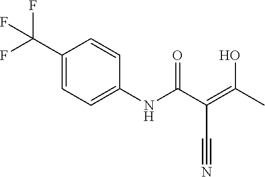 [SMILES: C/C(O)=C(\C#N)C(=O)Nc1ccc(C(F)(F)F)cc1]

### 2. Surface and Polymeric Stabilizers

The choice of one or more surface and polymeric stabilizers for nanoparticulate compositions of AP-325 is non-trivial and required extensive experimentation to realize a desirable formulation. Combinations of more than one surface and/or polymeric stabilizer can be used in the invention. Useful surface stabilizers which can be employed in the invention include, but are not limited to, known organic and inorganic pharmaceutical excipients. Such excipients include various polymers, low molecular weight oligomers, natural products, and surfactants. Surface stabilizers include nonionic, cationic, ionic, and zwitterionic compounds whereas polymeric stabilizers include well-known polymers widely used in pharmaceutical compositions like cellulose derivatives, polysaccharides, polyethylene derivatives, phospholipids, alginates and the like.

Representative examples of surface and polymeric stabilizers include, but are not limited to, hydroxypropyl methylcellulose, hydroxypropylcellulose, sodium lauryl sulfate, gelatin, casein, lecithin (phosphatides), bile salts like taurocholate and derived salts like sodium glyocholate, dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, the commercially available Tweens such as e.g., Tween 20® and Tween 80®, polyethylene glycols, polyoxyethylene stearates, colloidal silicon dioxide, phosphates, carboxymethylcellulose calcium, carboxymethylcellulose sodium, methylcellulose, hydroxyethylcellulose, hydroxypropylmethylcellulose phthalate, noncrystalline cellulose, magnesium aluminium silicate, triethanolamine, polyvinyl alcohol (PVA), poloxamers (e.g., Pluronics F68® and F127®, which are block copolymers of ethylene oxide and propylene oxide); poloxamines (e.g., Tetronic 908®, also known as Poloxamine 908®, which is a tetrafunctional block copolymer derived from sequential addition of propylene oxide and ethylene oxide to ethylenediamine, Tritons X-200® which is an alkyl aryl polyether sulfonate; Crodestas F-110®, which is a mixture of sucrose stearate and sucrose distearate, n-alkyl-beta-D-glucopyranosides; PEG-phospholipid, PEG-cholesterol, PEG-cholesterol derivative, PEG-vitamin A, PEG-vitamin E e.g. TPGS-1000®, lysozyme, and the like.

### 3. Other Pharmaceutical Excipients

Pharmaceutical AP-325 nanoparticulate compositions according to the invention may also comprise one or more binding agents, filling agents, lubricating agents, suspending agents, sweeteners, flavoring agents, buffers, wetting agents, disintegrants, effervescent agents, and other excipients. Such excipients are known in the art.

Suitable examples for starter pellets for fluid bed coating or granulation processes are lactose monohydrate, microcrystalline cellulose or isomalt.

Examples of filling agents and diluents are mannitol, lactose monohydrate, lactose anhydrous, and various starches, dibasic calcium phosphate, saccharides and/or mixtures of any of the foregoing; examples of binding agents are various celluloses and cross-linked polyvinylpyrrolidone, microcrystalline cellulose, such as Avicel® PH101 and Avicel® PH102, and silicified microcrystalline cellulose (ProSolv SMCCT™).

Suitable lubricants, including agents that act on the flowability of a powder to be compressed, are colloidal silicon dioxide, such as Aerosil® 200, talc, stearic acid, magnesium stearate, calcium stearate, and silica gel.

Suitable disintegrants include lightly crosslinked polyvinyl pyrrolidone, corn starch, potato starch, maize starch and modified starches, croscarmellose sodium, cross-povidone, sodium starch glycolate, and mixtures thereof.

Examples of effervescent agents are effervescent agents such as an organic acid and a carbonate or bicarbonate. Suitable organic acids include, for example, citric, tartaric, malic, fumaric, adipic, succinic, and alginic acids and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent agent may be present. Examples of sweeteners are any natural or artificial sweetener, such as sucrose, xylitol, sodium saccharin, cyclamate, aspartame, and acesulfame

### 4. Nanoparticulate AP-325 Active Particle Size Distribution

The compositions of the invention include particles of one or several of the active ingredients, e.g. AP-325, which have an effective average particle size of less than about 2000 nm (i.e., 2 microns). In other embodiments of the invention, said particles have an effective average particle size of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, as measured by light-scattering methods, microscopy, or other appropriate methods.

The effective average particle size is preferably less than 1400 nm, more preferably less than 900 nm, even more preferably less than 300 nm.

The effective average particle size is preferably more than 40 nm, more preferably more than 50 nm, even more preferably more than 60 nm.

The effective average particle size is preferably in the range from about 70 nm to about 220 nm, more preferably in the range from about 90 nm to about 210 nm, even more preferably in the range from about 100 nm to about 200 nm.

By “an effective average particle size of less than about 2000 nm” it is meant that at least 50% of said active agent particles have a particle size, by weight (volume based), of less than the effective average particle size when measured by the above-noted techniques, e.g., 50% of the particles have a size, by weight, of less than about 2 microns (or less than about 1900 nm, less than about 1800 nm, etc.).

In an advantageous embodiment, the particles are measured with a state-of-the-art analytical technique like a static or dynamic laser diffraction method (e.g. Malvern Sizer or Zeta Sizer). For example, the use of laser light diffraction to measure particle size is a widely known technique. Laser diffraction is a particle sizing method which uses the average relative angular intensity of scattered light. Instruments that use laser light diffraction to measure particle size have been available for many years from a number of different manufacturers. All laser diffraction instruments use the same basic method to measure particle size. All laser diffraction instruments require a beam of monochromatic light with a very uniform wave front. This beam of laser light is directed at the sample particles to be measured. When the light hits the particles, the light is diffracted or scattered from the particles. Detectors are used to measure the relative average intensity of the light scattered at various angles from the sample material. Once the relative intensity of light scattered at several different angles from the particles is known, the particle size and size distribution can be calculated.

5. Concentration of Nanoparticulate AP-325 and Active and Surface and/or Polymeric Stabilizer

The relative amounts of nanoparticulate AP-325 and one or more surface and/or polymeric stabilizers can vary widely. The optimal amount of the individual components can depend, for example, upon the hydrophilic lipophilic balance (HLB), melting point, pH dependent solubility, pKa values.

The concentration of AP-325 can vary from about 99.5% to about 0.001%, from about 95% to about 0.1%, or from about 90% to about 0.5%, by weight, based on the total combined dry weight of AP-325 and at least one surface and/or polymeric stabilizer, not including other excipients.

The concentration of at least one surface and/or polymeric stabilizer can vary from about 0.5% to about 99.9%, from about 5.0% to about 99.9%, or from about 10% to about 99.5%, by weight, based on the total combined dry weight of AP-325 and at least one surface and/or polymeric stabilizer, not including other excipients.

### D. Methods of Making Nanoparticulate AP-325 Compositions

### 1. Milling to Obtain Nanoparticulate AP-325 Compositions

Milling of AP-325 to obtain a nanoparticulate composition comprises dispersing particles of AP-325 in a liquid dispersion media in which AP-325 is poorly soluble, followed by applying mechanical means in the presence of hard grinding media to reduce the particle size of AP-325 to the desired effective average particle size. The dispersion media can be, for example, water, glycerine, polyethylene glycol (PEG), or glycol. Water is the preferred dispersion media.

The AP-325 particles are preferably reduced in size in the presence of at least one surface and/or polymeric stabilizer. Other compounds, such as a diluent, can be added to the AP-325 surface and polymeric stabilizer composition during the particle size reduction process. Dispersions can be manufactured continuously or in a batch mode.

### E. Methods of Using the Nanoparticulate AP-325 Compositions of the Invention

### 1. Treatment Applications

The AP-325 compositions of the invention are useful in treating and/or preventing, among other diseases and conditions, neuropathic pain and central nervous system trauma related disorders in humans.

The following examples are given to illustrate the present invention. It should be understood, however, that the invention is not limited to the specific conditions or details described in these examples.

### F. Examples

### 1. General Milling Conditions

The purpose of these examples was to show the application of the invented milling process for AP-325 and its derivatives in the laboratory scale to obtain fast results with limited amounts of active ingredient (screening phase) and in the pilot scale to demonstrate the suitability of the invented process for the manufacture of clinical trials samples.

Lab scale trials: The indicated amount of water (e.g. purified water) was weighed into a small grinding vessel made of zircon oxide (approx. 45 ml volume). Afterwards the given amounts of surfactant and stabilising polymer were added under stirring at ambient temperature until the components fully dissolved. Afterwards the described amount of AP-325 or its derivatives was slowly added under stirring to give an almost homogenous suspension. The magnetic bar was removed and an appropriate amount of yttria-stabilised zirconium milling beads in a suitable diameter (0.1-1.0 mm) was added to the suspension. The milling vessel was tightly closed and fixed in the laboratory nanomill chamber (e.g. Pulverisette 7 from Fritsch). Milling was started at an appropriate milling speed (e.g. 600-800 rpm) over an appropriate time interval (2-10 h) in a reverse mode function. After milling, the milling chamber was opened with care and the nanosuspension separated from the milling beads. The isolated nanosuspension was measured on particle size distribution (PSD) by a static laser diffraction (e.g. Malvern Mastersizer). Alternatively, the trials have been performed using a dual centrifugation system (e.g. Zentrimix 380) with 1 ml sample volume, with the same diameter range of milling beads and with a milling speed of about 1500-2500 rpm over a period between 1-4 hours.

Pilot scale trials: Initially the given amount of surfactant (e.g. sodium glycocholate) was dissolved in water (e.g. purified water) under stirring at ambient temperature. Afterwards the polymeric stabilizer (e.g. poloxamer 407) was added and fully dissolved in this solution. To get a homogeneous suspension, the AP-325 was incorporated step by step into the surfactant/polymer solution until all agglomerates of AP-325 were destroyed and a homogenous micro-suspension was obtained. Afterwards the micro-suspension was filled into the milling chamber of a suitable bead mill (e.g. Netzsch DeltaVita 300) and an appropriate amount of yttriastabilised zirconium milling beads (diameter from 0.1-1.0 mm) was added and the wet bead milling process was performed until the particle size distribution had reached the final requested nanometer range (several hours depending on the scale). A typical scale consisted of about 190 g of AP-325, about 1650 g of water, about 38 g polymeric stabilizer (e.g. Kolliphor P 407) and about 15 g of surface stabilizer (e.g. sodium glycocholate).

### 2. Examples for Nanoparticulate Compositions (Nanosuspensions) of AP-325 and its Derivatives

Nanosuspensions of AP-325 were prepared in accordance to section F1—Lab scale trials:

TABLE 1

| | | | Amount | | Amount | Amount | Amount | |
|---|---|---|---|---|---|---|---|---|
| | Type of | Nature of | surfactant | Type of | polymer | AP-325 | water | Total |
| No. | surfoctant | surfactant | [%] | polymer | [%] | [%] | [%] | [%] |
| N005 | TPGS-1000 | nonionic | 4.0 | HPMC | 2.0 | 10 | 84.00 | 100 |
| | | | | (Pharmacoat | | | | |
| | | | | 603) | | | | |
| N012 | TPGS-1000 | nonionic | 2.0 | HPMC | 2.0 | 10 | 86.00 | 100 |
| | | | | (Pharmacoat | | | | |
| | | | | 603) | | | | |
| N014 | Sodium | ionic | 0.8 | Poloxamer 407 | 2.0 | 10 | 87.20 | 100 |
| | glycocholate | | | (Lutrol F127) | | | | |
| N039 | Sodium | ionic | 0.6 | Poloxamer 407 | 2.0 | 10 | 87.40 | 100 |
| | glycocholate | | | (Lutrol F127) | | | | |
| N040 | Sodium | ionic | 0.6 | Poloxamer 188 | 4.0 | 10 | 85.40 | 100 |
| | glycocholate | | | (Lutrol F68) | | | | |
| N041 | Sodium | ionic | 0.6 | Poloxamer 407 | 4.0 | 10 | 85.40 | 100 |
| | glycocholate | | | (Lutrol F127) | | | | |
| N042 | Sodium | ionic | 0.8 | Poloxamer 407 | 2.0 | 10 | 87.20 | 100 |
| | taurocholate | | | (Lutrol F127) | | | | |
| N043 | Sodium | ionic | 0.8 | Poloxamer 188 | 2.0 | 10 | 87.20 | 100 |
| | taurocholate | | | (Lutrol F68) | | | | |
| N045 | Sodium | ionic | 0.8 | Poloxamer 188 | 4.0 | 10 | 85.20 | 100 |
| | taurocholate | | | (Lutrol F68) | | | | |
| N055 | Sodium | ionic | 0.8 | Poloxamer 188/ | 1.0/2.56 | 10 | 85.64 | 100 |
| | taurocholate | | | Phospholipon 90 | | | | |
| N057 | TPGS-1000 | nonionic | 2.0 | Poloxamer 188 | 4.0 | 10 | 84.00 | 100 |
| | | | | (Lutrol F68) | | | | |

Nanosuspensions of (Z)-2-cyano-3-hydroxy-N-[4-(trifluoromethyl)phenyl]hept-2-en-6-ynamide/FK778/Manitimus were prepared in accordance to section F1 Lab scale trials:

TABLE 2

| | | | Amount | | Amount | Amount | Amount | |
|---|---|---|---|---|---|---|---|---|
| | Type of | Nature of | surfactant | Type of | polymer | AP-325 | water | Total |
| No. | surfactant | surfactant | [%] | polymer | [%] | [%] | [%] | [%] |
| L001 | Sodium | ionic | 0.8 | Poloxamer 407 | 2.0 | 10 | 87.20 | 100 |
| | glycocholate | | | (Lutrol F127) | | | | |
| L002 | TPGS-1000 | nonionic | 4.0 | HPMC | 2.0 | 10 | 84.00 | 100 |
| | | | | (Pharmacoat | | | | |
| | | | | 603) | | | | |

Nanosuspension of 2-cyano-3-cyclopropyl-N-(4-fluorophenyl)-3-hydroxyacrylamide were prepared in accordance to section F1 Lab scale trials:

TABLE 3

| | | | Amount | | Amount | Amount | Amount | |
|---|---|---|---|---|---|---|---|---|
| | Type of | Nature of | surfactant | Type of | polymer | AP-325 | water | Total |
| No. | surfactant | surfactant | [%] | polymer | [%] | [%] | [%] | [%] |
| L003 | Sodium | ionic | 0.8 | Poloxamer 407 | 2.0 | 10 | 87.20 | 100 |
| | glycocholate | | | (Lutrol F127) | | | | |
| L004 | TPGS-1000 | nonionic | 4.0 | HPMC | 2.0 | 10 | 84.00 | 100 |
| | | | | (Pharmacoat | | | | |
| | | | | 603) | | | | |

Nanosuspensions of AP-325 were prepared in accordance to F1 Pilot scale trials:

TABLE 4

| | | | Amount | | Amount | Amount | Amount |
|---|---|---|---|---|---|---|---|
| | Type of | Nature of | surfactant | Type of | polymer | AP-325 | water |
| No. | surfactant | surfactant | [%] | polymer | [%] | [%] | [%] |
| N058 | Sodium | ionic | 0.8 | Poloxamer 407 | 2.0 | 10 | 87.20 |
| | glycocholate | | | (Lutrol F127) | | | |
| N071 | Sodium | ionic | 0.8 | Poloxamer 407 | 2.0 | 10 | 87.20 |
| | glycocholate | | | (Lutrol F127) | | | |
| N072 | Sodium | ionic | 0.8 | Poloxamer 407 | 2.0 | 10 | 87.20 |
| | glycocholate | | | (Lutrol F127) | | | |
| N073 | Sodium | ionic | 0.8 | Poloxamer 407 | 2.0 | 10 | 87.20 |
| | glycocholate | | | (Lutrol F127) | | | |
| N076 | Sodium | ionic | 0.8 | Poloxamer 407 | 2.0 | 10 | 87.20 |
| | glycocholate | | | (Lutrol F127) | | | |
| N077 | Sodium | ionic | 0.8 | Poloxamer 407 | 2.0 | 10 | 87.20 |
| | glycocholate | | | (Lutrol F127) | | | |

### 3. Particle Size Distribution of Nanosuspensions Containing AP-325 and Derivatives, Stability and Prove of Crystallinity

Particle size distribution was measured by laser light scattering (e.g. Malvern Mastersizer) for the nanosuspensions of AP-325 obtained under F2 and Table 1:

TABLE 5

| No. | D10 [μm] | D50 [μm] | D 90 [μm] |
|---|---|---|---|
| N005 | 0.07 | 0.12 | 0.20 |
| N012 | 0.07 | 0.12 | 0.21 |
| N014 | 0.07 | 0.14 | 0.91 |
| N039 | 0.07 | 0.14 | 1.11 |
| N040 | 0.07 | 0.13 | 0.54 |
| N041 | 0.07 | 0.14 | 0.81 |
| N042 | 0.08 | 0.13 | 0.30 |
| N043 | 0.08 | 0.13 | 0.23 |
| N045 | 0.08 | 0.14 | 0.28 |
| N055 | 0.07 | 0.14 | 1.25 |
| N057 | 0.07 | 0.14 | 1.05 |

Particle size distribution was measured by laser light scattering (e.g. Malvern Mastersizer) for the nanosuspensions of (Z)-2-cyano-3-hydroxy-N-[4-(trifluoromethyl)phenyl]hept-2-en-6-ynamide/FK778/Manitimus obtained under F2 and Table 2

TABLE 6

| No. | D10 [nm] | D50 [nm] | D90 [nm] |
|---|---|---|---|
| L001 | 81 | 271 | 1911 |
| L002 | 71 | 133 | 317 |

Particle size distribution was measured by laser light scattering (e.g. Malvern Mastersizer) for the nanosuspensions of 2-cyano-3-cyclopropyl-N-(4-fluorophenyl)-3-hydroxyacrylamide obtained under F2 and Table 3:

TABLE 7

| No. | D10 [nm] | D50 [nm] | D90 [nm] |
|---|---|---|---|
| L003 | 27 | 117 | 532 |
| L004 | 24 | 95.2 | 1210 |

Particle size distribution was measured by laser light scattering (e.g. Malvern Mastersizer) for the nanosuspensions of AP-325 obtained under F2 and Table 4:

TABLE 8

| No. | D10 [μm] | D50 [μm] | D90 [μm] |
|---|---|---|---|
| N058 | 0.07 | 0.13 | 0.29 |
| N071 | 0.07 | 0.14 | 0.34 |
| N072 | 0.07 | 0.14 | 0.44 |
| N073 | 0.07 | 0.15 | 0.43 |
| N076 | 0.07 | 0.15 | 0.48 |
| N077 | 0.07 | 0.15 | 0.47 |

Particle size distribution (PSD) was measured by laser light scattering (e.g. Malvern Mastersizer) for the nanosuspensions of AP-325 obtained under F2 and Table 4 and have been measured again after certain time intervals and storage conditions (temperature/humidity). The results confirmed that the PSD was unchanged over the investigated time intervals and conditions confirming the good stability of the nanoparticulate composition of AP-325.

Condition: 2-8° C. (refrigerator)/Batch N077

TABLE 9

| | | | |
|---|---|---|---|
| No. | D10 [μm] | D50 [μm] | D90 [μm] |

| | | | | |
|---|---|---|---|---|
| 1 | week | 0.07 | 0.15 | 0.52 |
| 2 | weeks | 0.07 | 0.15 | 0.49 |
| 4 | weeks | 0.07 | 0.15 | 0.49 |
| 12 | weeks | 0.07 | 0.15 | 0.50 |

Condition: 25° C./60% r.h. (climate chamber)/Batch N077

TABLE 10

| | | | |
|---|---|---|---|
| No. | D10 [μm] | D50 [μm] | D90 [μm] |

| | | | | |
|---|---|---|---|---|
| 1 | week | 0.07 | 0.17 | 0.95 |
| 2 | weeks | 0.07 | 0.17 | 0.87 |
| 4 | weeks | 0.07 | 0.17 | 0.91 |
| 12 | weeks | 0.07 | 0.16 | 0.88 |

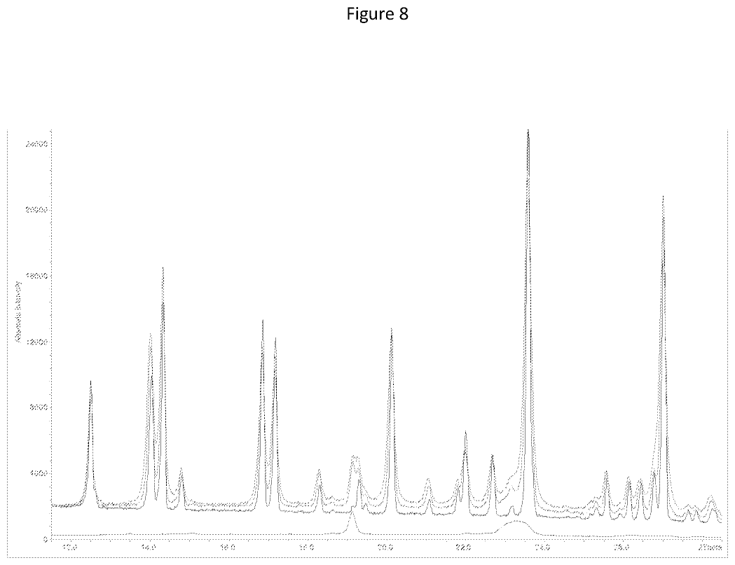

It is important that during the milling process no change of the crystallinity of the active ingredient occurs (e.g. change of a polymeric form). To prove that the crystallinity of the active ingredient remained unchanged, a part of suspension of N073 had been dried and analysed by powder X-ray diffraction (PXRD; e.g. STOE Stadi P Transmission mode, Cu—K alpha irradiation) against the pure active ingredient, the placebo mixture and the active ingredient suspended and dried. As it can be seen in FIG. 8 all X-ray patterns remained unchanged after the milling process, proving that the crystallinity of the active ingredient AP-325 has not changed.

It is further important that the obtained nanoparticulate compositions of AP-325 are stable in the acidic environment as otherwise the “nanoeffect” would simply disappear if the nanoparticles would be converted into microcrystals above 2000 nm under such conditions. In order to prove the stability in acidic environment the nanosuspensions of AP-325 have been treated with 0.06 M hydrochloric acid (pH=1.2) over a certain period (e.g. 1-2 hours) at 37° C. The data shown in table 11 prove the good stability of the invented nanosuspensions of AP-325 under acidic conditions:

TABLE 11

| Batch No. | D10 [μm] | D50 [μm] | D90 [μm] |
|---|---|---|---|
| N014 | 0.07 | 0.14 | 0.79 |
| N022 (=N012) | 0.07 | 0.12 | 0.21 |
| N024 (=N005) | 0.07 | 0.12 | 0.20 |

Degradation of the active ingredient may occur during the milling process. In order to prove that no significant degradation of AP-325 was obtained the nanosuspension has been investigated after milling by a specific, validated HPLC method (e.g. using a HPLC Agilent 1290 instrument). The results in table 12 prove that no degradation could be detected after the milling process, all impurities remained at a very low level:

TABLE 12

| Batch | N005 | N012 | N014 |
|---|---|---|---|
| Colour of | white | white | white |
| content | | | |
| Purity | | | |
| COPR1 | — | — | — |
| (RRT 0.50) | | | |
| COPR2 | — | 0.05% | 0.05% |
| (RRT 0.57) | | | |
| unknown | — | 0.05% | — |
| (RRT 0.67) | | | |
| unknown | — | — | — |
| (RRT 0.86) | | | |
| Sum of | — | 0.10% | 0.05% |
| impurities | | | |

Investigations had also been made to detect residual traces of zirconium and yttrium from the milling beads collision during the milling process confirming that only very low traces in the low ppm range for both elements were obtained in the nanosuspensions of AP-325 indicating the suitability of the invented milling process for its intended use.

### 4. Examples for Granulate Compositions Containing Nanoparticles of AP-325

The nanosuspension of AP-325 obtained under F1 Pilot scale trials were converted into a dosage form in which the nanoparticles are bound on a suitable carrier in order to prevent the so called Ostwald ripening effect of the nanoparticles in the nanosuspension itself. The compositions shown are only one possibility of converting the nanoparticles into a powder formulation and other techniques like spray drying, extrusion, direct granulation may be used as well.

Fluid Bed Layering: To the nanosuspensions obtained under F1 Pilot scale trials: a suitable polymeric stabilizer is added under stirring at ambient temperature until fully dissolved. Further, the completed layering suspension containing AP-325 was layered on s suitable carrier in a fluid bed coater (e.g. Unilab from Bosch with bottom spray nozzles). For the layering process standard nozzles and filters have been used, the inlet temperature was set to max. 60° C. to get a product temperature of about 40° C. The spraying pressure was set less than 1 bar. A typical scale consisted of about 280 g Nanosuspension obtained under F1 Pilot scale trials, about 9 g of additional polymeric stabilizer (e.g. Hypromellose 6 mPas) and about 1220 g of carrier (e.g. Isomalt galenIQ 960). The process was completed within several hours, depending on the scale.

Final blending: The final dried pellets from [58] were blended with suitable lubricants for several minutes in a suitable blender at ambient temperature to avoid sticking during the capsule filling process. A typical scale consisted of about 800 g pellets containing nanoparticles of AP-325 and about 18 g of a lubricant (e.g. Talc).

Encapsulation: The mixture is filled into hard gelatine capsules (e.g. size 0) by pellet dosing unit of the capsule filling machine (e.g. Bonapace capsule filling machine). Table 13 gives an overview of different batches which had been produced in accordance to this section:

TABLE 13

| | | Amount | | Filling | |
|---|---|---|---|---|---|
| | Granule | Granules | Amount | weight | |
| Batch | batch | [mg] | Talc | [mg] | Dose [mg] |
| K001 | C012 | 214.8 | 5.7 | 220.5 | 5 |
| K002 | C012 | 107.4 | 2.5 | 109.9 | 2.5 |
| K003 | C010 | 237.8 | 5.0 | 242.8 | 25 |
| K004 | C011 | 214.6 | 5.0 | 219.6 | 25 |
| K006 | C014 | 219.4 | 5.0 | 224.4 | 5 |
| K007 | C014 | 109.7 | 2.5 | 112.2 | 2.5 |

Composition of a capsule: Typical compositions of capsules formulations in accordance to this invention are represented in tables 14 and 15:

TABLE 14
One capsule contains (based on a theoretical assay
of 100% granules) for the 25 mg strength:

| Ingredient | Quantity | |
|---|---|---|
| AP-325 | 25.00 | mg |
| Poloxamer 407 | 5.00 | mg |
| Sodium glycocholate | 2.00 | mg |
| Hydroxypropylmethylcellulose E6 (6 mPas) | 8.00 | mg |
| Isomalt | 218.00 | mg |
| Talc | 5.00 | mg |

TABLE 15
One capsule contains (based on a theoretical assay
of 100% granules) for the 5 mg strength:

| Ingredient | Quantity | |
|---|---|---|
| AP-325 | 5 | mg |
| Poloxamer 407 | 1.00 | mg |
| Sodium glycocholate | 0.40 | mg |
| Hydroxypropylmethylcellulose E6 (6 mPas) | 1.60 | mg |
| Isomalt | 218.00 | mg |
| Talc | 5.00 | mg |

### 5. Particle Size and Dissolution Profiles of Nanoparticulate AP-325 Composition in Hard Gelatine Capsules

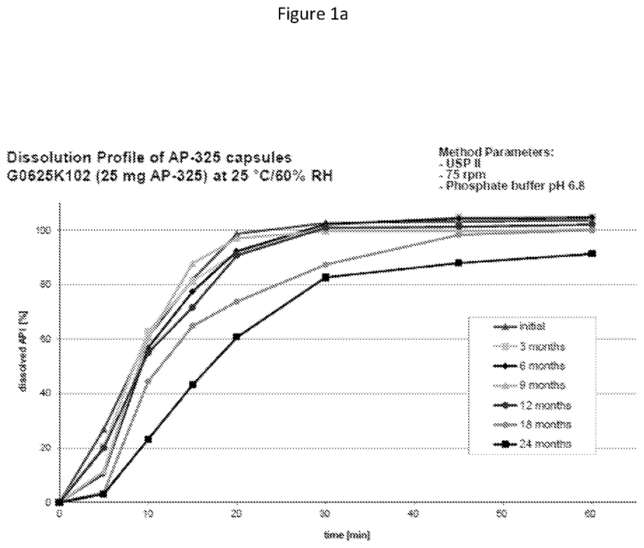

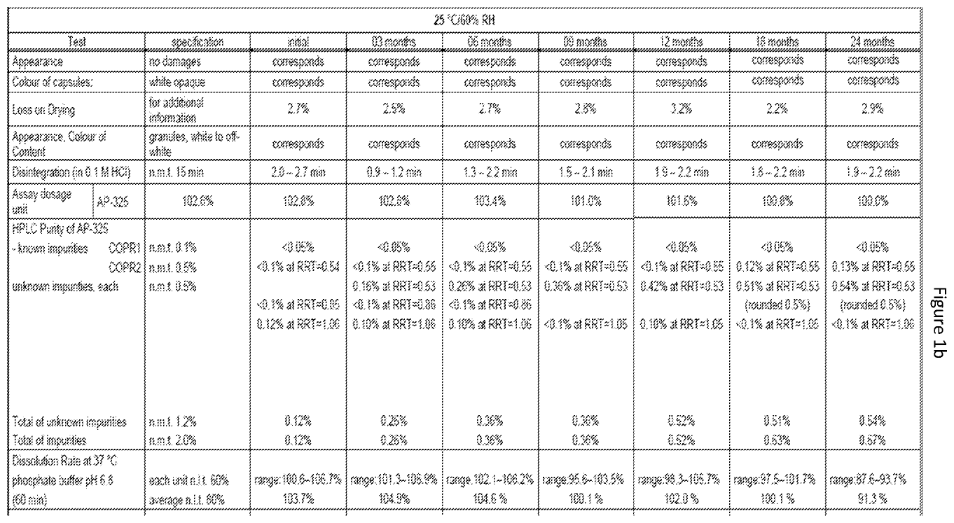

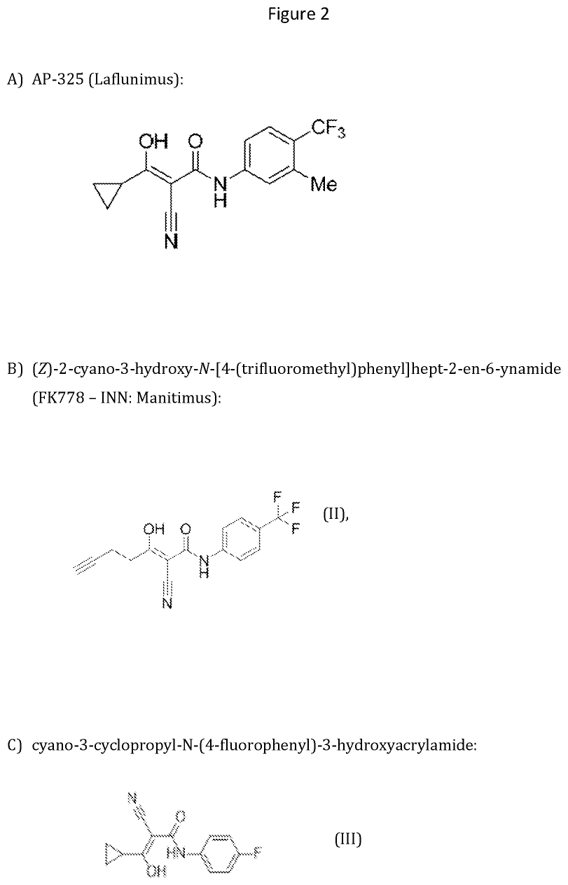

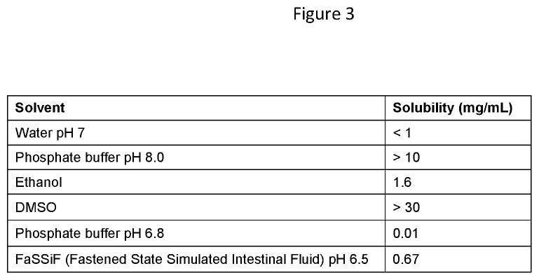

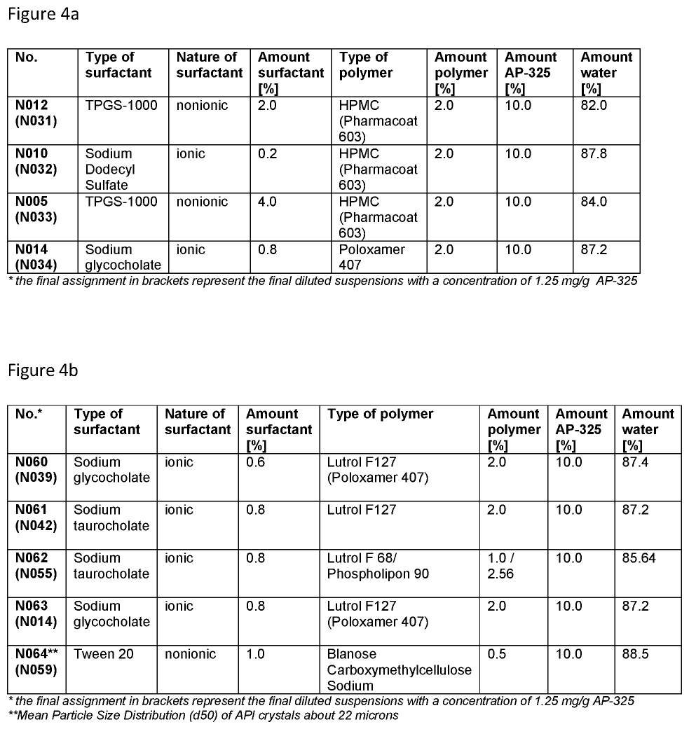

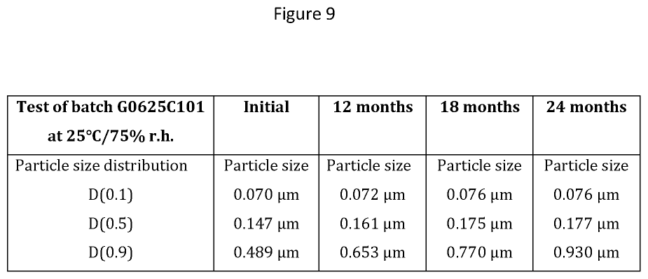

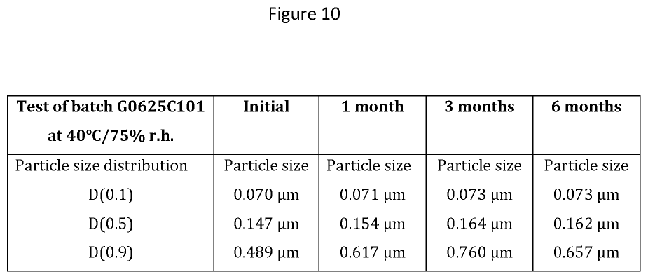

Important parameters for nanoparticulate compositions during the stability testing under different conditions are that the particle size of the nanoparticles of AP-325 and that the derived dissolution profiles in physiological media remained unchanged. FIG. 9 and FIG. 10 show the particle size behaviour during stability testing at different conditions. As a water insoluble lubricant (talc) had been used in the formulations the particle size measurements had been done for the granules of the corresponding batch as the lubricant is interfering with the PSD measurements with the laser light scattering techniques. The results demonstrate that the PSD remained unchanged at storage conditions of 25° C./60% r.h. and 40° C./75% r.h. over a period of 24 respective 6 months representing a very good stability of the nanoparticles of AP-325 in the dosage form. This good stability is also confirmed by the dissolution data presented in FIG. 1a over a period of 24 months at 25° C./60% r.h at 37° C. in physiological phosphate buffer. The small drop of the dissolution profile at 18 and 24 months is obviously related to a small change (e.g. sticking) of the granules within the capsules, but the overall release is still well above 80% after 60 minutes and fulfils the relevant requirements of the Ph. Eur. for oral dosage forms.

### 6. Stability Data of Nanoparticulate AP-325 Composition in Hard Gelatine Capsules

The stability parameters PSD and dissolution have been already described in [063], but further data are as well important to represent a good stability of a dosage form. These stability data are summarized in FIG. 1b and demonstrate that the AP-325 nanoparticulate composition is unaffected under the described ICH storage conditions to any changes of the investigated parameters and can be regarded as very stable over the investigated storage period.

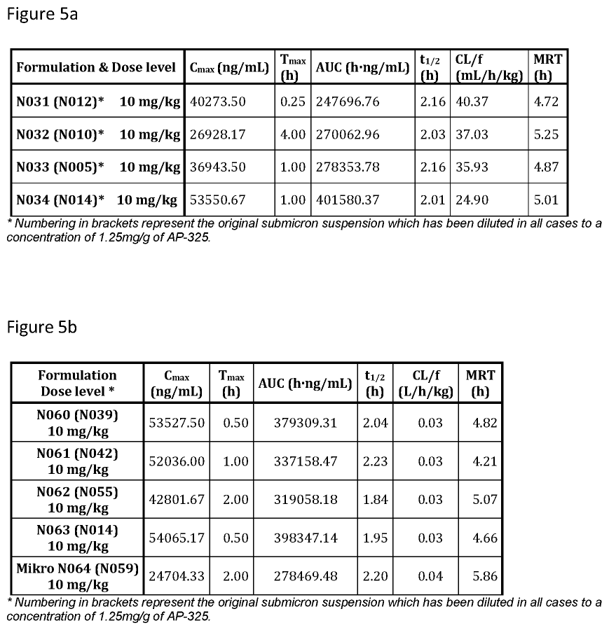

### 7. Rat Study Protocol (Pk Study, See Also FIGS. 5a) and b)

### Application

p.o. (gavage) as single dose

### Animal Specification

Species: Sprague Dawley rats, fastened

Sex: Males

### Experimental Groups and Doses

Animals were treated with a single dose according to table 16a and 16b:

TABLE 16a
Experimental groups and doses (study 1)

| | | | | | Administration |
|---|---|---|---|---|---|
| | | No. and sex | Route of | Dose | volume |
| No. | Group | of animals | administration | [mg/kg] | [ml/kg] |
| 1 | N031 | 8 males | oral | 10 | 3 |
| 2 | N032 | 8 males | oral | 10 | 3 |
| 3 | N033 | 8 males | oral | 10 | 3 |
| 4 | N034 | 8 males | oral | 10 | 3 |

TABLE 16b
Experimental groups and doses (study 2)

| | | No. and sex | Route of | Dose | Administration |
|---|---|---|---|---|---|
| No. | Group | of animals | administration | [mg/kg] | volume [ml/kg] |
| 1 | N060 | 8 males | oral | 10 | 2 |
| | (N039) | | | | |
| 2 | N061 | 8 males | oral | 10 | 2 |
| | (N042) | | | | |
| 3 | N062 | 8 males | oral | 10 | 2 |
| | (N055) | | | | |
| 4 | N063 | 8 males | oral | 10 | 2 |
| | (N014) | | | | |
| 5 | N064 | 8 males | oral | 10 | 2 |
| | (N059) | | | | |

The total volume to be administered was calculated according to the individual body weight recorded on the day of administration. Blood sampling was performed at several time points within 24 h.

Blood samples approx. 600 μl were withdrawn using a butterfly and capillaries from the tail vein. The collected blood was immediately transferred into lithium heparin-containing tubes (e.g. Saarstedt), shaken by hand and stored for 30 minutes on crushed ice until centrifugation (2.500×g and 4° C. for 10 minutes). The supernatant plasma was separated and transferred into pre-labeled plastic tubes. The plasma samples were stored in an ultra-freezer (−80° C.) until shipment

### Analysis

For the pharmacokinetic studies of AP-325 in rats, a specific HPLC-MS/MS method was developed and validated for the quantification of AP-325 in rat plasma.

### 8. Dog Study Protocol (See Also FIG. 6)

### Application

p.o. as single dose

### Animal Specification

Species: Beagle dogs

Sex: 4 Males each

### Experimental Groups and Doses

Group: 2

Dose: 100 mg/animal

Blood samples were withdrawn, and the collected blood was immediately transferred into lithium heparin-containing tubes, shaken by hand and stored for 30 minutes on crushed ice until centrifugation (2.500×g and 4° C. for 10 minutes). The supernatant plasma was separated and transferred into pre-labeled plastic tubes. The plasma samples were stored in an ultra-freezer (−80° C.) until shipment.

### Analysis

For the pharmacokinetic studies of AP-325 in dogs, a specific HPLC-MS/MS method was developed and validated for the quantification of AP-325 in dog plasma.

### 9. Phase 1 Study Protocol Humans (FIG. 7)

Application: single dose of 5, 15, 40, 100 and 150 mg of AP-325 (nanoparticulate composition)

Design: randomised, placebo controlled, double blind

Subjects: Healthy white male subjects between 18-45 years; BMI with 18 to 29.9 kg/m²; body weight 70 kg

### Analysis

For the pharmacokinetic studies of AP-325 in humans, a specific HPLC-MS/MS method was developed and validated for the quantification of AP-325 in human plasma.

The invention relates to a nanoparticulate of AP-325 or a AP-325 derivative composition comprising: (a) particles of AP-325 or its derivative having an effective average particle size of less than about 2000 nm; and (b) at least one surface and/or polymeric stabilizer.

Furthermore, the invention relates to a pharmaceutical composition comprising said composition in combination with at least one pharmaceutically acceptable excipient.

Furthermore, the invention relates to the use of said pharmaceutical composition for preparation of a medicament.

Moreover, the invention relates to a method of making a nanoparticulate AP-325 or an AP-325 derivative composition comprising contacting particles of AP-325 or an AP-325 derivative with at least one surface and/or polymeric stabilizer for a time and under conditions sufficient to provide a composition comprising AP-325 or a AP-325 derivative particles having an effective average particle size of less than about 2 microns.

A further aspect of the invention is a system for the treatment of certain diseases in a subject comprising administering to a subject of an effective amount of a composition comprising (a) particles of AP-325 or derivatives thereof having an average effective particle size of less than about 2000 nm, and (b) at least one surface and/or polymeric stabilizer. The nanoparticulate composition in this system can have a reduced Tmax, a higher cₘₐₓ and higher AUC in mammalian subjects compared to a composition containing AP-325 or one of its derivatives having an average effective particle size of more than about 2000 nm. In this system the nanoparticulate composition does not have a disintegration time of less or equal to 3 minutes. The system may further comprise one or more active agents useful for the treatment of certain human diseases.

1. A nanoparticulate composition comprising:
(a) particles comprising at least one active ingredient, wherein the particles have an effective average particle size is in the range of about 70 nm to about 220 nm measured by laser light scattering method, wherein at least 50% of said active agent particles have a particle size, by weight (volume based), of less than the effective average particle size; and
(b) at least one surface stabilizer and/or at least one polymeric stabilizer, wherein the composition comprises:
(aa) particles of at least one active ingredient selected from the group consisting of (Z)-2-cyano-3-cyclopropyl-3-hydroxy-N-(3-methyl-4-(trifluoromethyl)phenyl) prop-2-enamide, (Z)-2-cyano-3-hydroxy-N-[4-(trifluoromethyl)phenyl]hept-2-en-6-ynamide, 2-cyano-3-cyclopropyl-N-(4-fluorophenyl)-3-hydroxyacrylamide, derivatives thereof, salts thereof and pro-drugs thereof, wherein the particles have an effective average particle size of less than about 2000 nm; and
(bb) at least one surface stabilizer and/or at least one polymeric stabilizer.

2. The composition of claim 1, wherein the effective average particle size is in the range from about 90 nm to about 210 nm, optionally in the range from about 100 nm to about 200 nm,

3. The composition of claim 1, wherein the at least one active ingredient is present in an amount in the range from about 99.5% by weight to about 0.001% by weight, optionally in the range from about 95% by weight to about 0.1% by weight, and optionally in the range from about 90% by weight to about 0.5% by weight, based on the total combined dry weight of the active ingredient and the at least one surface stabilizer and/or polymeric stabilizer, not including other excipients.

4. A pharmaceutical composition comprising a composition according to claim 1, in combination with at least one pharmaceutically acceptable excipient.

5. A method of making a composition of claim 1 comprising contacting particles of the at least one active ingredient with at least one surface stabilizer and/or at least one polymeric stabilizer for a time and under conditions sufficient to provide a composition comprising particles of the active ingredient having the effective average particle size.

6. A method of making a solid oral dosage form in which the nanoparticles from the nanosuspensions containing one or several of the active ingredients as claimed in claim 1 are bound on a suitable pharmaceutical excipient or carrier by using a fluid bed drying process, a spray drying process, an extrusion process or a granulation process.

7. A system for use in the treatment or the prevention of neuropathic pain and/or central nervous system trauma related disorder and/or certain other diseases in a subject comprising administering to a subject of an effective amount of the pharmaceutical composition of claim 4.

8. The system for use of claim 7, wherein the nanoparticulate composition has a reduced Tₘₐₓ, a higher cₘₐₓ and higher AUC in mammalian subjects compared to a composition containing the one or several active ingredients as claimed in claim 1 having an average effective particle size of more than about 2000 nm.

9. The system for use of claim 7, wherein the nanoparticulate composition does not have a disintegration time of less or equal to 3 min.

10. The system for use of claim 7, further comprising one or more additional active agents useful for the treatment of certain human diseases.

11. The system for use of claim 7, wherein the system is used for the treatment of wherein the compound is used in the treatment of peripheral and/or predominantly peripheral neuropathic pain or central and/or predominantly central neuropathic pain.

12. The system for use of claim 7, wherein the system is used to treat an inflammatory disease, type I diabetes and/or type II diabetes.
